(19) United States
(12) Patent Application Publication
(10) Pub. No.: US 2009/0203940 A1
(43) Pub. Date: Aug. 13, 2009

(54) Method for preparing 4-[2-(dimethylamino)ethoxy]benzylamine as itopride-hydrocloride salt mediate

(76) Inventors: Dong Yeon Kim, Seoul (KR); Jae G. Kim, Gyeonggi-do (KR); Dae Jin Cho, Seoul (KR); Gong Y. Lee, Gyeonggi-Do (KR); Hong Y. Kim, Seoul (KR); Seok H. Woo, Gyeonggi-do (KR)

Correspondence Address: EDWARDS ANGELL PALMER & DODGE LLP, P.O. BOX 55874, BOSTON, MA, 02205, US

(21) Appl. No.: 11/658,746

(22) PCT Filed: Sep. 15, 2004
(86) PCT No.: PCT/KR04/02342

§ 371 Date: Oct. 3, 2008

(30) Foreign Application Priority Data
Jul. 28, 2004 (KR) 10-2004-0058972

(51) Int. Cl. C07C 213/06

(52) U.S. Cl. 564/347

(57) ABSTRACT

The present invention relates to a novel method for preparing an itopride-hydrochloride mediate of the formula 1, which is useful for digestive tract activator, and more particularly, the present invention provides with a method for preparing 4-[2-(dimethylamino)ethoxy]benzylamine of the formula 1, an itopride-hydrochloride mediate, whereby being capable of manufacturing in a high yield and lowering cost through a simple purification and a selective reaction, and the method is harmless and safe to human and environment due to not generating toxic gas. And specially, a super-high pressure hydrogenation and a reduction reaction using a metal catalyst are not carried out, therefore, it is very safe method, and also special manufacturing equipments are not needed.

### TECHNICAL FIELD

The present invention relates to a method for preparing 4-[2-(dimethylamino)ethoxy]benzylamine as an itopride-hydrochloride salt mediate of the following formula 1, which is useful for a digestive tract motility activator, and more particularly, to a method for preparing an itopride-hydrochloride salt mediate, which comprises the step of esterification with the following formula 2 and formula 3 as a starting material (if R₁ is methylamine, R₂ is F, Cl, Br or I, R₃ is OH); or the step of carrying out esterification and reduction reaction simultaneously (if R₁ is CN, R₂ is OH, R₃ is F, Cl, Br or I), whereby being capable of a mass synthesis in high-yield and low cost through a simple manufacturing process and a selective reaction, and this method is harmless and safe to human and environment.

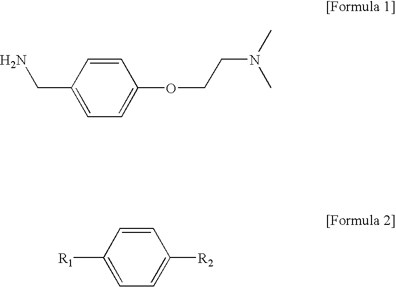

where R₁ is CN or CH₂NH₂, R₂ is OH, F, Br, Cl or I.

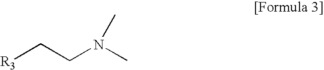

where R₃ is F, Cl, Br, I or OH.

Specially, the present invention includes all the processes for the manufacture of itopride-hydrochloride salt mediate by using the formula 2 and formula 3 compounds as a starting material.

### BACKGROUND ART

Itopride-hydrochloride salt, a digestive tract motility activator, is a useful drug for improving the Non-ulcer Dyspepsia Symptom of digestive system such as gastric discomfort, abdominal distension or the like.

A couple of digestive tract motility activator, including

4-amino-5-chloro-N-[(2-diethylamino)ethyl]-2-methoxybenzamide (called as metoclopramide, Merck Index, 13th edition, 6164) of the following formula 4, is known.

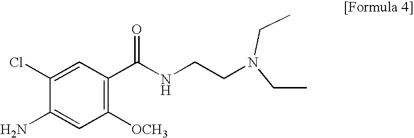

However, a conventional digestive tract motility activator has problems in effect and safety. Specially, cisapride[PROPULSID^{?}, Yanssen, Merck Index, 13th edition, 2340] has an excellent effect, but the production thereof has been stopped due to adverse reaction by a ventricular arrhythmia when administrating together with imidazole or macrolide antibiotics. Accordingly, itopride-hydrochloride salt having an excellent effect and safety is very useful drug. Specially, there is no adverse reaction.

### DISCLOSURE OF INVENTION

Technical Problem

Korean Patent Laid-Open Publication No. 1989-0005036 discloses a method for preparing itopride-hydrochloride salt of the following Reaction Scheme 1.

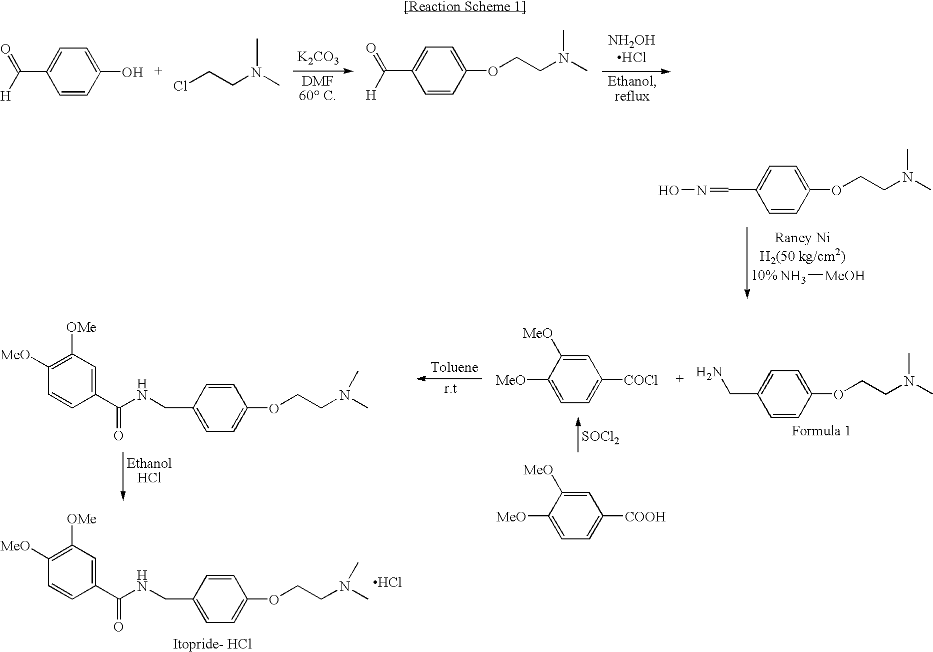

In the above reaction scheme 1, the method for preparing formula 1 comprises three steps. However, there are problems as follows.

In the first step, purification using distillation is required, so that a lot of purification time and distillation equipments are needed. Specially, in the third step, preparation of 4-[2-(dimethylamino)ethoxy]benzylamine of formula 1 from 4-[2-(1-dimethylamino)ethoxy]benzaldoxime as a starting material, Raney nickel, a metal catalyst, having a high flammability, is used in an amount of ⅛˜ 1/9 against the weight of the initiator, so that it is very dangerous (reduction reaction), and also after reaction, it is very hard to treat the residue of Raney nickel.

Specially, there is possibility of explosiveness due to using hydrogen in super-high pressure state (50 kg/cm²), so that an equipment to secure safety is needed. In addition, using ammonia gas, saturated in methanol, as a reaction solvent causes to generate toxic gas, which brings on serious problems to human and environmental pollution.

Also, in Korean Patent Laid-Open Publication No. 1989-0005036, a yield according to the individual steps is not disclosed, so that it is difficult to confirm the total reaction yield. And special manufacturing equipments for the three-step processes are required and it takes long time for purification, so that it takes long manufacturing time, and the costs of production of a high purity mediate is high.

As described above, in a conventional method for synthesizing itopride-hydrochloride salt mediate, there is possibility of explosiveness due to using Raney nickel, a metal catalyst, having a high-flammability with hydrogen in super-high pressure state, so that an equipment to secure safety is needed. In addition, using ammonia gas saturated in methanol causes to generate toxic gas, which brings on serious problems to human and environmental pollution. And also, it takes long manufacturing time, and the costs of production of a high purity mediate are high.

Technical Solution

The present invention is provided to solve the problems of conventional technology as described above.

An object of the present invention is to provide a method for preparing an itopride-hydrochloride salt mediate, which comprises the step of esterification with the following formula 2 and formula 3 as a starting material (if R₁ is methylamine, R₂ is F, Cl, Br or I, R₃ is OH); or the step of carrying out esterification and reduction reaction simultaneously (if R₁ is CN, R₂ is OH, R₃ is F, Cl, Br or I), whereby providing a high purity formula 1 compound in high yield and low cost through a simple manufacturing process and a selective reaction, and this method is harmless and safe to human and environment.

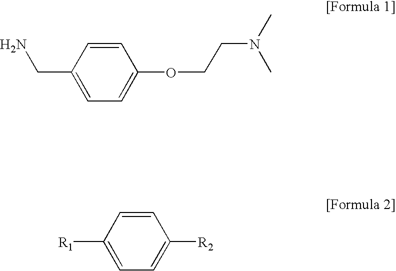

where R₁ is CN or CH₂NH₂, R₂ is OH, F, Br, Cl or I.

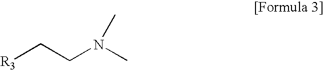

where R₃ is F, Cl, Br, I or OH.

To accomplish the above objects, a method for preparing itopride-hydrochloride salt mediate according to the present invention can manufacture the formula 1 compound through a manufacturing process comprising a single esterification with the following formula 2 and formula 3 compounds as a starting material.

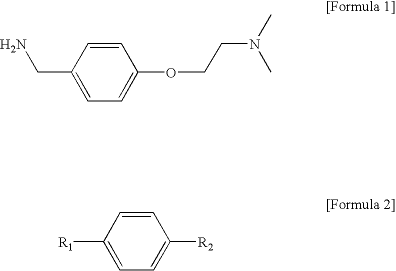

where R₁ is CN or CH₂ NH₂, R₂ is OH, F, Br, Cl or I.

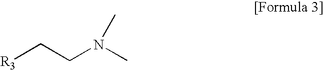

where R₃ is F, Cl, Br, I or OH.

The above formula 1 compound can be prepared by the above esterification comprising the steps of mixing 1.2 to 5.0 equivalents of the formula 3 compound, based on 1.0 equivalent of the formula 2 compound, in the presence of 1.1 to 2.0 equivalents of a base at a temperature of 120 to 170° C., wherein the base is selected from the group consisting of sodium hydride, potassium hydride calcium hydride, pyridine, triethylamine, potassium hydroxide, sodium hydroxide, potassium carbonate and sodium carbonate; dropwising the formula 2 compound thereto, followed by mixing them at a temperature of 120 to 170° C.; and extracting the formula 1 compound with a solvent selected from the group consisting of chloroform, dichloromethane and ethylacetate.

The R₁ of formula 2 is CH₂NH₂, R₂ is halogen, and R₃ is hydroxy.

The above manufacturing process including the esterification to prepare the formula 1 compound comprises the steps of dropwising 1.2 to 1.8 equivalents of the formula 3 compound, based on 1.0 equivalent compound of the formula 2, in the presence of 1.1 to 2.0 equivalents of a base selected from the group consisting of sodium hydride, calcium hydride, potassium hydride, potassium hydroxide, sodium hydroxide, potassium carbonate, sodium carbonate, pyridine and triethylamine, followed by refluxing and mixing at room temperature for 0.5 to 2 hours, in which the formula 3 compound is dissolved in solvent selected from the group consisting of N,N-dimethylformamide, dimethylsulfoxide, acetone and dichloromethane; dropwising the formula 2 compound, followed by refluxing and mixing it for 0.5 to 2 hours; extracting with a solvent selected from the group consisting of chloroform, dichloromethane and ethylacetate; dissolving the extract in a solvent, ethanol or methanol, followed by dropwising 0.05 to 0.2 equivalents of metal catalyst selected from the group consisting of cobalt(II)sulfate-7 hydrate, copper(II)sulfate-5 hydrate, copper(II)chloride-2 hydrate, sellium(II)chloride, cobalt(II)chloride, titanium(II)chloride and samarium(II)chloride, and then mixing it for 20 to 40 minutes; dropwising 3.0 to 5.0 equivalents of sodium borohydride, followed by refluxing and mixing for 15 to 25 hours; and extracting the formula 1 compound with a solvent selected from the group consisting of chloroform, dichloromethane and ethylacetate.

In the formula 2, R₁ is CN, R₂ is hydroxy, and R₃ is halogen.

### BEST MODE FOR CARRYING OUT THE INVENTION

Hereinafter, the present invention will be illustrated in more detail as follows.

A manufacture of itopride-hydrochloride salt mediate can prepare the following formula 1 through a simple process with the following formula 2 and formula 3 as a starting material such as the following reaction process (reaction formula 2, reaction formula 3).

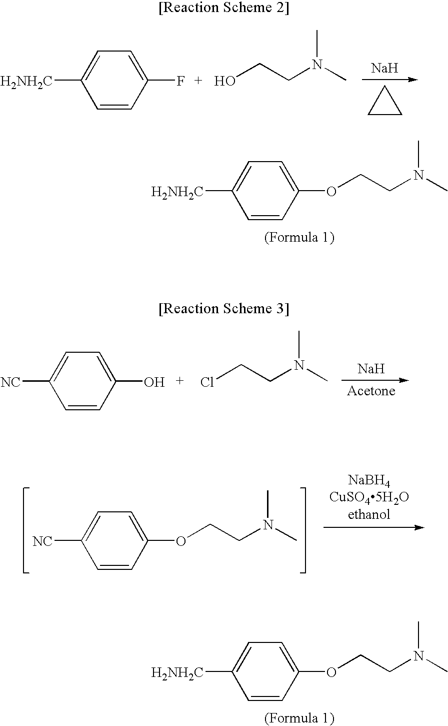

In the reaction scheme 2 according to the present invention, 4-[2-(dimethylamino)ethoxy]benzylamine, which is the formula 1 compound (itopride-hydrochloride mediate), can be prepared through a single process of esterification with 4-fluorobenzylamine and 2-(dimethylamino)ethanol as a starting material. The amount of 2-(dimethylamino)ethanol used is 1.2 to 5.0 equivalents, based on 1.0 equivalent of 4-fluorobenzylamine, preferably 1.7 equivalents. And also, the base used is preferably sodium hydride, and the equivalent thereof is 1.1 to 2.0, based on 1.0 equivalent of 4-fluorobenzylamine, preferably 1.4 equivalents.

In the reaction scheme 3 according to the present invention, 4-[2-(dimethylamino)ethoxy]benzylamine, which is the formula 1 compound (itopride-hydrochloride mediate), can be prepared by carrying out simultaneously esterification and reduction reaction with 4-hydroxy benzonitrile and 2-(dimethylamino)ethyl chloride as a starting material.

The amount of 2-(dimethylamino)ethyl chloride used is 1.2 to 1.8 equivalents, based on 1.0 equivalent of 4-hydroxy benzonitrile, preferably 1.5 equivalents.

As a metal catalyst used in reduction reaction of the formula 3, copper(II)sulfate-5 hydrate and sodium borohydride are simultaneously used, and the equivalent of copper(II)sulfate-5 hydrate is 0.05 to 0.2 equivalents, preferably 0.1 equivalents. The equivalent of sodium borohydride is 3.5 to 5.5 equivalents, preferably 5.0 equivalents.

As compared to a conventional method, a method according to the present invention is a simple process, and it takes short purification time, and a hydrogen reduction reaction using a metal catalyst in super-high pressure (50 kg/cm²) is not needed. Therefore, a high purity itopride-hydrochloride salt mediate (formula 1 compound) can be prepared very safely with low cost.

### MODE FOR THE INVENTION

Hereinafter, the present invention will be described in detail referring to the following examples. However, the examples according to the present invention can be modified in other forms, and the scope of the present invention is not limited to the following examples.

### Example 1

### Preparation of 4-[2-(dimethyl amino)ethoxy]benzylamine

2.54 g (63.43 mmol) of 60% sodium hydride was slowly dropwised to 6.63 g (74.37 mmol) of 2-(dimethylamino)ethanol at 0° C.

After finishing the dropwise, the temperature of reactor was raised to 130˜140° C. and mixed for 1 hour. 5.48 g (43.79 mmol) of 4-fluorobenzylamine was slowly dropwised therein, followed by mixing at 130˜140° C. for 5 hours. After finishing the reaction, the reactant was cooled down to room temperature. 100 ml of H₂O was added thereto, followed by mixing for 30 minutes, and then extracted with chloroform (150 ml×2), and dried with magnesium sulfate anhydrous, which was then filtered, and 7.74 g (91% yield) of a desired product was obtained by decompressing-distilling.

¹HNMR(CDCl₃, ppm): 1.63(br,NH₂), 2.31(s,6H), 2.67˜2.72(t,2H), 3.77(s,2H), 4.00˜4.05(t,2H), 6.84˜6.89(d,2H), 7.17˜7.21(d,2H)

### Example 2

### Preparation of 4-[2-(dimethyl amino)ethoxy]benzonitrile

20 g (168 mmol) of 4-hydroxybenzonitrile was dissolved in 200 ml of acetone, and 34.8 g (251.8 mmol) of potassium hydroxide was added thereto, which was then refluxed and mixed for 1 hour.

36.3 g (251.8 mmol) of 2-(dimethylamino)ethyl chloride was slowly dropwised to the reactant, followed by refluxing and mixing for 8 hours.

The reactant was cooled down to room temperature, and acetone was removed by decompression and concentration. Thereafter, 300 ml of dichloromethane was extracted, which was then dried with magnesium sulfate anhydrous, and then 31 g (97% yield) of a desired product was obtained by decompression and concentration.

¹HNMR(CDCl₃,ppm): 2.30(s,6H), 2.71˜2.74(t,2H), 4.06˜4.09(t,2H), 6.92˜6.96(d,2H), 7.52˜7.56(d,2H)

### Example 3

### Preparation of 4-[2-(dimethylamino)ethoxy]benzylamine: reduction reaction by the formula 3

2 g (10.5 mmol) of 4-[2-(dimethylamino)ethoxy]benzonitrile was dissolved in 30 ml of ethanol, and 0.23 (0.92 mmol) of copper(II)sulfate-5 hydrate (2 mol aqueous solution) was added thereto. And 1.74 g (45.94 mmol) of sodium borohydride was slowly dropwised, followed by refluxing and mixing for 20 hours.

The reactant was cooled down to room temperature, and extracted with ethylacetate, and dried with magnesium sulfate anhydrous, and then 1.63 g (80% yield) of a desired product was obtained by decompression and concentration.

¹HNMR(CDCl₃,ppm): 1.63(br,NH₂), 2.30(s,6H), 2.66˜2.71(t,2H), 3.77(s,2H), 4.01˜4.06(t,2H), 6.83˜6.88(d,2H), 7.15˜7.20(d,2H)

### INDUSTRIAL APPLICABILITY

A method for preparing itopride-hydrochloride salt mediate, digestive tract motility activator, according to the present invention has the advantages of high yield through a selective reaction, and low cost and high purity product through simple-fast purification method, and a harmless and safe method to human and environment.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

1. A method for preparing itopride-hydrochloride salt mediate, which manufactures the formula 1 compound through a manufacturing process comprising a single esterification with the following formula 2 and formula 3 compounds as a starting material. 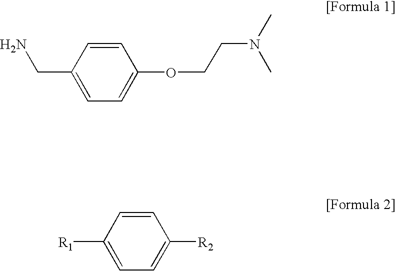
where R₁ is CN or CH₂NH₂, R₂ is OH, F, Br, Cl or I. 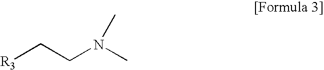
where R₃ is F, Cl, Br, I or OH.

2. The method for preparing itopride-hydrochloride salt mediate according to claim 1, wherein the above formula 1 compound can be prepared by the above esterification, comprising the steps of:
mixing 1.2 to 5.0 equivalents of the formula 3 compound based on 1.0 equivalent of the formula 2 compound in the presence of 1.1 to 2.0 equivalents of a base at a temperature of 120 to 170° C., wherein the base is selected from the group consisting of sodium hydride, potassium hydride, calcium hydride, pyridine, triethylamine, potassium hydroxide, sodium hydroxide, potassium carbonate, and sodium carbonate;
dropwising the formula 2 compound thereto, followed by mixing them at a temperature of 120 to 170° C.; and
extracting the formula 1 compound with a solvent selected from the group consisting of chloroform, dichloromethane and ethylacetate.

3. The method for preparing itopride-hydrochloride salt mediate according to claim 2, wherein in the formula 2, R₁ is CH₂NH₂, R₂is halogen, and R₃ is hydroxy.

4. The method for preparing itopride-hydrochloride salt mediate according to claim 1, wherein the above manufacturing process including the esterification to prepare the formula 1 compound comprises the steps of:
dropwising 1.2 to 1.8 equivalents of the formula 3 compound, based on 1.0 equivalent compound of the formula 2, in the presence of 1.1 to 2.0 equivalents of a base selected from the group consisting of sodium hydride, calcium hydride, potassium hydride, potassium hydroxide, sodium hydroxide, potassium carbonate, sodium carbonate, pyridine and triethylamine, followed by refluxing and mixing at room temperature for 0.5 to 2 hours, in which the formula 3 compound is dissolved in solvent selected from the group consisting of N,N-dimethylformamide, dimethylsulfoxide, acetone and dichloromethane;
dropwising the formula 2 compound, followed by refluxing and mixing it for 0.5 to 2 hours;
extracting with a solvent selected from the group consisting of chloroform, dichloromethane and ethylacetate;
dissolving the extract in a solvent, ethanol or methanol, followed by dropwising 0.05 to 0.2 equivalents of metal catalyst selected from the group consisting of cobalt(II)sulfate-7 hydrate, copper(II)sulfate-5 hydrate, copper(II)chloride-2 hydrate, sellium(II)chloride, cobalt(II)chloride, titanium(II)chloride and samarium(II)chloride, and then mixing it for 20 to 40 minutes;
dropwising 3.0 to 5.0 equivalents of sodium borohydride, followed by refluxing and mixing for 15 to 25 hours; and
extracting the formula 1 compound with a solvent selected from the extracting the formula 1 compound with a solvent selected from the extracting the formula 1 compound with a solvent selected from the group consisting of chloroform, dichloromethane and ethylacetate.

5. The method for preparing itopride-hydrochloride salt mediate according to claim 4, wherein in the formula 2, R₁ is CN, R₂ is hydroxy, and R₃ is halogen.
